(19) United States
(12) Patent Application Publication
(10) Pub. No.: US 2018/0161246 A1
(43) Pub. Date: Jun. 14, 2018

(54) COMPRESSED VAPORIZER TABLET AND METHOD

(71) Applicant: Compressed Perforated Puck Technologies Inc., Edmonton (CA)

(72) Inventor: David DAVIS, Edmonton (CA)

(73) Assignee: Compressed Perforated Puck Technologies Inc., Edmonton, AB (CA)

(21) Appl. No.: 15/576,610

(22) PCT Filed: May 20, 2016
(86) PCT No.: PCT/CA2016/000152

§ 371 Date: Nov. 22, 2017

Related U.S. Application Data
(60) Provisional application No. 62/165,705, filed on May 22, 2015.

(51) Int. Cl. A61J 3/10; A61K 36/185; A61K 45/06; A61K 9/20; A61M 11/04; A61M 15/00

(52) U.S. Cl. CPC A61J 3/10; A61K 36/185; A61K 45/06; A61K 9/2072; A61M 2209/06; A61M 15/0028; A61J 2205/10; A61M 2205/6072; A61M 11/04

(57) ABSTRACT

There is disclosed a compressed vaporizer tablet and method. In an embodiment, the method comprises: obtaining one or more plant source materials containing one or more active medicinal ingredients; compressing the one or more plant source materials under pressure and heat into a tablet; and forming one or more through holes in the tablet, whereby the through holes provide increased tablet surface area during vaporization. The tablet may further include one or more ribbed edges to further increase the tablet surface area. The tablet is preferably individually sealed in a blister pack to increase shelf life of the tablet, and to provide safety and convenience features for use.

### FIELD

The present disclosure relates to compressed tablets, and more generally to a compressed medicinal tablet for vaporizers.

### BACKGROUND

Many medicinal ingredients of therapeutic efficacy for various conditions or ailments may be found in plant sources. In some cases, delivering these medicinal ingredients from the plant source may involve subjecting the plant source material to combustion in order to release the active ingredients in the plant source material for inhalation. While conventional methods such as lighting and inhaling the resulting vapor of the burning plant source material may provide effective delivery of the active ingredients, there may also be adverse side effects resulting from formation of toxic compounds in the gaseous and airborne particles in the smoke formed from combustion. Such toxins in the smoke may include neurotoxins which may be poisonous or destructive to nerve tissue of the individuals inhaling them. There may also be other toxins which have the potential to damage the respiratory system or cardiovascular system of these individuals when this delivery method is used repeatedly over a long period of time.

What is therefore needed is an improved technological solution that overcomes at least some of these limitations.

### SUMMARY

The present disclosure relates to a compressed vaporizer tablet which contains processed plant source material which allows the medicinal ingredients to be released through vaporization, utilizing a heat vaporizer, without the need to subject the plant source material to combustion.

In an aspect, the compressed vaporizer tablet comprises a tablet formed by compressing loose plant source material that has been processed into a compressible state. This processing may involve drying, shredding, grinding, and mixing the plant source with one or more base materials which may help the loose plant source material and any base material to bind together during compression, helping the resulting compressed tablet retain its shape.

In an embodiment, each tablet is compressed with compression molds having at least one post or core which produces a hole in the compressed tablet. A plurality of such posts or cores may be spaced apart in the mold in order to form a pattern of a plurality of holes in the compressed tablet.

In an embodiment, the holes formed in each tablet are through holes which pass through the tablet, such that each hole provides an open cavity passing through the tablet. The size, number, and pattern of through holes may be selected to provide varying rates of vaporization. Generally speaking, a larger number of holes will provide a greater surface area, resulting in an increased rate of vaporization of the compressed plant source material.

In an embodiment, rather than forming the holes during compression, the holes may be formed in a subsequent, separate drilling operation which drills through the tablet to form the through holes. However, this may result in a loss of plant source material, and significantly increase processing time for forming the tablets.

In another embodiment, each tablet includes ribbed edges along the outer side of each tablet, so as to further increase the surface area of the tablet. Generally speaking, a larger number of ribs increases the rate of vaporization of the compressed plant source material.

In another embodiment, each tablet may include an alignment feature, such that the tablet may be aligned in a particular orientation within a vaporizer. This may be useful, for example, if the vaporizer includes a plurality of vents configured to align with the through holes formed in the tablet. By aligning these through holes with the plurality of vents in the vaporizer, greater control over the rate of vaporization of the compressed plant source material may be obtained.

In another embodiment, each tablet may include a particular pattern in the ribbed edges, which pattern may be detected by a vaporizer to determine a particular type of tablet, which may require different vaporizer settings, for example the temperature profile, or another parameter which controls the rate of vaporization of the tablet.

In another embodiment, the compressed vaporizer tablet may include a blend of different plant source materials selected to alleviate specific conditions or ailments. These blends may be selected based on the active ingredients found in each plant source material, and the amount of each plant source material in the blend is proportional to the desired proportion of active ingredients.

In an embodiment, the compressed vaporizer tablet may include various other active ingredients apart from the main plant source material, such that the compressed vaporizer tablet may deliver more than one active ingredient at the same time.

In an embodiment, the compressed vaporizer tablet may include various flavoring agents, such that the compressed vaporizer tablet may deliver different aromatic flavors.

In another embodiment, the compressed vaporizer tablet may be individually sealed and packaged in blister packs in order to increase the shelf life of the tablet. Sealing in a blister pack may strictly control the amount of desired humidity in the tablet, in order to maintain freshness and longevity of the ingredients.

In another embodiment, each tablet may include a label or a coating on at least one side of the tablet, which label or coating may include a barcode, or another type of machine readable code to identify the particular type of tablet. As a typical compressed vaporizer tablet in accordance with the present invention is significantly larger than typical pharmaceutical pills, tablets or capsules meant to be swallowed, the surface area of the compressed tablet is more than sufficient to provide a surface area suitable for including a label.

In this respect, before explaining at least one embodiment of the system and method of the present disclosure in detail, it is to be understood that the present system and method is not limited in its application to the details of construction and to the arrangements of the components set forth in the following description or illustrated in the drawings. The present system and method is capable of other embodiments and of being practiced and carried out in various ways. Also, it is to be understood that the phraseology and terminology employed herein are for the purpose of description and should not be regarded as limiting.

### BRIEF DESCRIPTION OF THE DRAWINGS

FIG. 1 shows a compressed vaporizer tablet including a plurality of holes in accordance with an illustrative embodiment.

FIG. 2 shows a compressed vaporizer tablet including ribbed edges in accordance with another illustrative embodiment.

FIG. 3 shows a compressed vaporizer tablet including a label or tag and one or more alignment features.

FIG. 4 shows an illustrative example of a compressed vaporizer tablet sealed within a blister pack.

### DETAILED DESCRIPTION

As noted above, the present disclosure relates to a compressed vaporizer tablet which contains processed plant source material which allows the medicinal ingredients to be released through vaporization, utilizing a heat vaporizer, without the need to subject the plant source material to combustion.

An illustrative embodiment of the platform will now be described in more detail with reference to the figures.

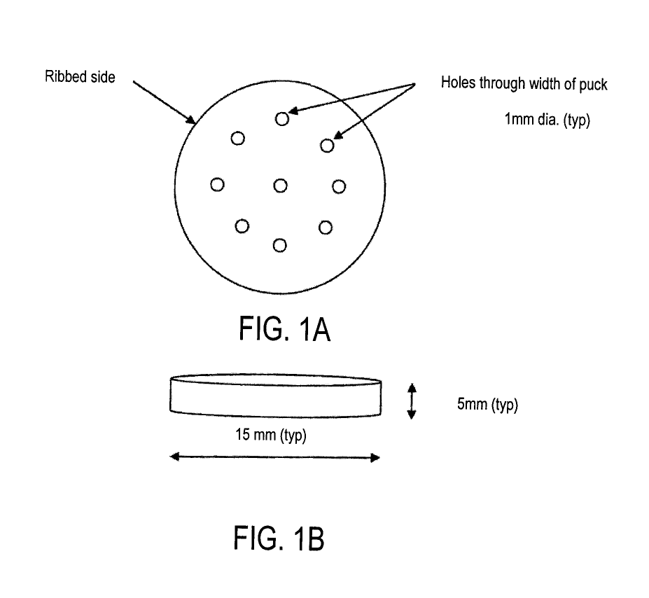

Referring to FIG. 1, shown is a compressed vaporizer tablet including a plurality of holes in accordance with an illustrative embodiment.

In an aspect, the compressed vaporizer tablet comprises a tablet formed by compressing loose plant source material that has been processed into a compressible state. This processing may involve drying, shredding, grinding, and mixing the plant source with one or more base materials which may help the loose plant source material and any base material to bind together during compression, helping the resulting compressed tablet retain its shape.

As an illustrative example, in an embodiment, the plant source material may be the hemp plant which is composed of approximately 20% lignin, a polymer in plants that provides rigidity. In an embodiment, as the plant source material is compressed, it is heated by frictional forces. The lignins (contained in all woody-cellulose materials) begin to flow and act as a natural glue to bind the compressed plant source materials. Sticky trichomes, which are present predominantly in flowers, will also serve to bind the material, possibly reducing the total pressure needed to form the tablets. When the compressed material exits the compression machine, the lignins cool, solidify and hold the plant source material together to form the tablet.

In experimentation, it has been found that hydraulic presses will generally produce compressed vaporizer tablets which are suitably dense and of sufficient hardness to retain their rigid shape. However, it will be appreciated that other types of presses (e.g. mechanical presses) may also be used if they can provide the sufficient compression force and desired ambient parameters.

During compression, temperatures rise sufficiently to make the raw material liberate various adhesives that will assist in keeping the particles together in the compressed shape. However, to make this process successful, the moisture content for most hemp based material is preferably around 10-12%, although the range may be somewhat broader in the range of 8-14% and still provide good performance.

In a preferred embodiment, inventor has found that compressing hemp and having it reach temperatures between 80° C. and 93° C. is beneficial, as this temperature boils off the toxin benzene making, the product safer to inhale. Furthermore, the inventor has realized that temperatures should not exceed 133° C., which is the boiling temperature of the flavinoid Beta-sitosterol. Although some of the following cannabinoids require temperatures above 200 degrees Celsius to evaporate, setting a vaporizer to that temperature runs the risk of causing combustion, which should be avoided.

In a preferred embodiment, tablets are formed using special dies. High pressures (e.g. 10 tons) and temperatures (e.g. 200° F./93° C.) are generated in this process, which softens components of the hemp (the lignin) and binds the material in the tablet together. No additional adhesives are required.

To form a tablet, the granulated material must be metered into a cavity formed by two punches and a die. A tablet is formed by the combined pressing action of two punches and a die. In the first step of a typical operation, the bottom punch is lowered in the die creating a cavity into which the ground material is fed. The exact depth of the lower punch can be precisely controlled to meter the amount of material that fills the cavity. The excess is scraped from the top of the die, and the lower punch is drawn down and temporarily covered to prevent spillage. Then, the upper punch is brought down into contact with the material as the cover is removed. The force of compression is delivered by high pressure compression rolls which fuse the ground material together into a hard tablet. After compression, the lower punch is raised to eject the tablet.

There are generally two types of tablet presses: single-punch and rotary tablet presses. Most high speed tablet presses take the form of a rotating turret that holds any number of punches. As they rotate around the turret, the punches come into contact with cams which control the punch's vertical position. Punches and dies are usually custom made for each application, and can be made in a wide variety of sizes, shapes, and can be customized with manufacturer codes and scoring lines to make tablets easier to break. Depending on tablet size, shape, material, and press configuration, a typical modern press can produce from 250,000 to over 1,000,000 tablets an hour.

By way of example, 1 gram of plant source material may be compressed into a generally cylindrical tablet of approximately 15 mm in diameter and 5 mm in height or thickness, as shown in FIG. 1. It will be appreciated that the amount of plant source material and the dimensions of the tablet are provided by way of illustration only, and are not meant to be limiting. For example, the compressed vaporizer tablet may be increased to 25 mm (approximately 1 inch) in diameter or even larger. Preferably, the thickness of the tablet may range from about 2 mm-6 mm, but the tablet may be thinner or thicker as may be desired.

The illustrative tablet shown in FIG. 1 further includes nine holes of approximately 1 mm in diameter. Again, this dimension is illustrative, and is not meant to be limiting. In this example, each hole is no more than about 4 mm from any other hole or an outside edge. This will mean that heat will have to penetrate no more than about 2 mm from any surface of the tablet, whether on the outside surface, or from an inner surface within one of the holes.

In an embodiment, each tablet is compressed with compression molds having at least one post or core which produces a hole in the compressed tablet. A plurality of such posts or cores may be spaced apart in the mold in order to form a pattern of a plurality of holes in the compressed tablet. The pattern of holes may be provided to align with vents provided in a vaporizer, such that the vaporization occurs more efficiently, as discussed further below.

In an embodiment, the holes formed in each tablet are through holes which pass through the tablet, such that each hole provides an open cavity passing through the tablet. The size, number, and pattern of through holes may be selected to provide varying rates of vaporization. Generally speaking, a larger number of holes will provide a greater surface area, resulting in an increased rate of vaporization of the compressed plant source material.

In an embodiment, rather than forming the holes during compression of the tablet, the holes may be formed in a subsequent drilling operation which drills through the compressed tablet to form the through holes. However, this additional step may result in a loss of plant source material through drilling, and significantly increase processing time for forming the tablets.

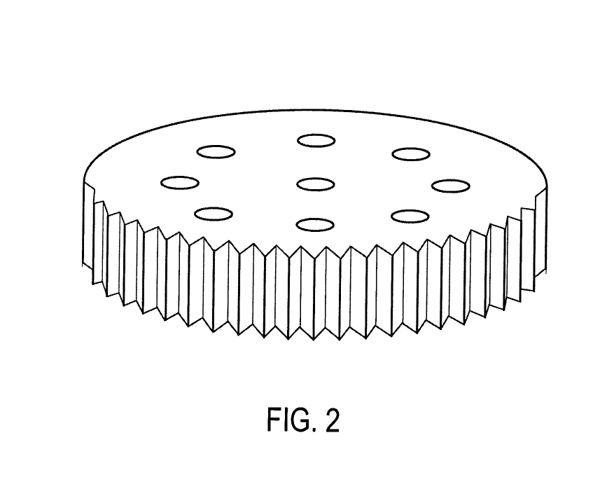

Now referring to FIG. 2, in another embodiment, each tablet includes ribbed edges along the side of each tablet, so as to further increase the surface area of the tablet. Generally speaking, a larger number of ribs increases the rate of vaporization of the compressed plant source material.

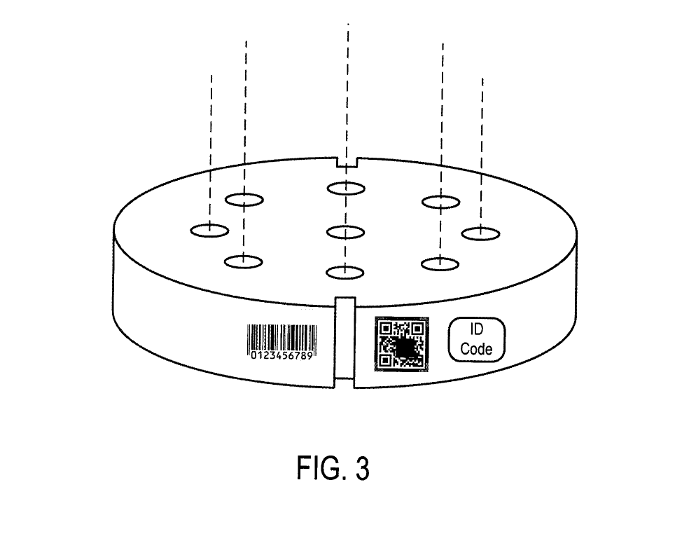

In another embodiment, as shown in FIG. 3, each tablet may include an alignment feature, such that the tablet may be aligned in a particular orientation within a vaporizer. This may be useful, for example, if the vaporizer includes a plurality of vents configured to align with the through holes formed in the tablet. By aligning these through holes with the plurality of vents in the vaporizer, greater control over the rate of vaporization of the compressed plant source material may be obtained.

In another embodiment, each tablet may include a particular pattern in the ribbed edges, which pattern may be detected by a vaporizer to determine a particular type of tablet, which may require different vaporizer settings, for example the temperature profile, or another parameter which controls the rate of vaporization of the tablet.

In another embodiment, each compressed vaporizer tablet may have a particular shape that has one or more distinct sides rather than a generally cylindrical shape. Differently shaped tablets may indicate different types of tablets, for example.

In another embodiment, the compressed vaporizer tablet may include a blend of different plant source materials selected to alleviate specific conditions or ailments. These blends may be selected based on the active ingredients found in each plant source material, and the amount of each plant source material in the blend is proportional to the desired proportion of active ingredients.

As an illustrative example, THC (tetrahydrocannabinol) and CBD (cannabidiol) are the two most prominent chemical compounds in the cannabis plant. Consequently, the vast majority of research to date has focused on the ratio of these two cannabinoids.

THC is helpful for treating many, many ailments. Studies have shown that it has medicinal benefits for ALS (Lou Gehrig's disease), Alzheimer's, anxiety, arthritis, chemotherapy side effects, Crohn's Disease, chronic pain, fibromyalgia, HIV-related peripheral neuropathy, Huntington's Disease, incontinence, insomnia, multiple sclerosis, pruritus, sleep apnea, and Tourette Syndrome, among others. THC has even been shown to kill cancerous tumors, and shown to be therapeutic in the treatment of nausea and appetite loss. THC has also been found to alleviate spasticity in patients with multiple sclerosis. THC has also been found effective in treating difficult-to-treat nerve pain commonly found in amputees, AIDS patients, and patients with multiple sclerosis.

CBD is the other major medicinal compound identified so far, and interest in its effects is growing. It is non-psychoactive. BD works through a number of complex mechanisms. Preclinical studies indicate that CBD has analgesic (pain-relieving), anticonvulsant, anti-psychotic and neuroprotective effects. Unlike THC, it does not bind to the CB1 or CB2 cannabinoids receptors, which is why it does not produce THC-like psychoactivity.

CBD is used to help with acne, ADD, anxiety, arthritis, chronic pain, depression, diabetes, Dravet syndrome, epilepsy, glaucoma, Huntington's Disease, multiple sclerosis, neuropathic pain, Parkinson's, and schizophrenia, just to name a few. CBD has also been shown to kill cancer cells.

Other important chemical compounds in the cannabis plant include THCA. Prior to drying, the chemical that becomes THC is known as THCA (tetrahydrocannibinolic acid). In its fresh form, THCA is non-psychoactive. Growing research is showing the benefits of juicing raw, fresh cannabis. It is believed that THCA has medicinal properties that are lost when the plant is dried, and it can be metabolized in much larger doses than THC, making it potentially more effective. THCA appears to help with chronic immune-system disorders, including potential treatment of chronic Lupus.

CBN (cannabinol) is another chemical compound found in the cannabis plant. CBN is created when THC is exposed to light and oxygen. It's known to have some mild psychoactive effects, and it appears to increase the effects of THC. CBN may make users dizzy or groggy, and is not usually sought-after for medicinal purposes.

CBC (cannabichromene) is another chemical compound found in the cannabis plant. Evidence suggests that CBC it may play a role in the anti-inflammatory and anti-viral effects of cannabis, and may contribute to the overall analgesic effects of medical cannabis. A 2011 study in the British Journal of Pharmacology found that CBD and CBC stimulated descending pathways of antinociception and caused analgesia by interacting with several target proteins involved in nociceptive control. It helps in fighting bacteria as an anti-fungal also as an anti-inflammatory, pain relief, anti-biotic, depression and brain growth.

### Ratios of Compounds

Recent research has been done into CBD:THC ratios by the pharmaceutical industry, specifically around the GW Pharmaceuticals' Sativex, which has a 1:1 ratio of THC and CBD. In the clinical trials phase of drug development, researchers examined the effects of THC, CBD, and combination extracts on sleep, pain control, and muscle spasms. They found that 1:1 CBD:THC extracts provided the most therapeutic relief across all categories.

CBD and THC combinations also show therapeutic promise across a number of disease states for which there has been limited therapeutic breakthrough to date. CBD modulates waking via activation of neurons in the hypothalamus and DRD. Both regions are apparently involved in the generation of alertness. Also, CBD increases DA levels as measured by microdialysis and HPLC procedures. Since CBD induces alertness, it might be of therapeutic value in sleep disorders such as excessive somnolence.

For example, in Amyotrophic Lateral Sclerosis (ALS), THC has been shown to delay motor deterioration and increase long term survival. Recent work has built on this study to show that the addition of CBD in conjunction with THC leads to a 14% increase in motor performance and an increase in survival beyond the survival rates with THC alone.

CBD and THC combinations have also been shown to increase alertness in some patients. By contrast, THC alone has more sedative effects. In fact, CBD is just an antagonist of CB1 receptors. It is probably true then that indica plants have a very high THC % compared to sativas and this has a stronger biphasic stimulant/sedative effect (CB1 receptors are the most widespread receptors in the brain, so its not unlikely that they can do both). The amount of CBD in most drug strains probably has no effect at all since CBD is not present in large amounts and has a much lower affinity for CB1 receptors compared to THC.

Illustrative examples of CBD to THC ratios include the following:

88:1—Non-psychoactive. Charlotte's Web is a well know example of a CBD dominant strain. Used to treat children with severe epilepsy and Dravet syndrome.

18:1—Non-psychoactive. Some patients find CBD dominant medicines helpful for anxiety, depression, psychosis and other mood disorders.

8:1—Non-psychoactive. Some patients find mid-range CBD:THC ratios helpful for spasms, convulsions, tremors, endocrine disorders, metabolic syndrome and overall wellness.

4:1—Borderline psychoactive. For patients who have some tolerance for THC. Some patients find mid-range ratios helpful for pain relief, immune support and other health benefits. Has been found to kill all forms of cancer cells in a petri dish.

2:1—Psychoactive in larger doses. For patients who have some tolerance for THC. Some patients find balanced ratios helpful for inflammation, chronic pain, gastrointestinal issues and stress relief.

1:1—Psychoactive. For patients who tolerate THC well. Some patients find a balanced ratio helpful for neuropathic pain, rheumatism and overall mood enhancement.

### Dosing

A patient's sensitivity to THC (tetrahydrocannabinol) is a key factor to determining the appropriate ratio and dosage of high CBD cannabis medicine. CBD can lessen or neutralize the intoxicating effects of THC. So a greater ratio of CBD-to-THC means less of a “high.” But CBD-dominant cannabis remedies with little THC, while not intoxicating, are not necessarily the most effective therapeutic option. That's because CBD and THC heighten one another's medicinal effects. A combination of CBD and THC will likely have a greater anti-cancer effect or analgesic (painkilling) effect, for example, than CBD or THC alone.

Whether smoking or using a vaporizer, the general guideline is to start low and increase dosage incrementally. Patients should start with a very low dose and stop therapy if any undesirable or unacceptable effects occur. Patients should also wait between inhalations for a few minutes to gauge the strength of the effects.

Several surveys have shown that the average dose of medical marijuana is 1 to 3 g/day when smoked or vaporized. In one recent Canadian study, 25 mg of pharmaceutical-grade cannabis with a THC (delta-9-tetrahydrocannabinol) content of 9.4% was effective in reducing intensity of pain, improved sleep and was well tolerated when smoked as a single inhalation 3×/day for five days.

In an embodiment, the compressed vaporizer tablet may include various other active ingredients apart from the main plant source material, such that the compressed vaporizer tablet may deliver more than one active ingredient at the same time.

In an embodiment, the compressed vaporizer tablet may include various flavoring agents, such that the compressed vaporizer tablet may deliver different flavors.

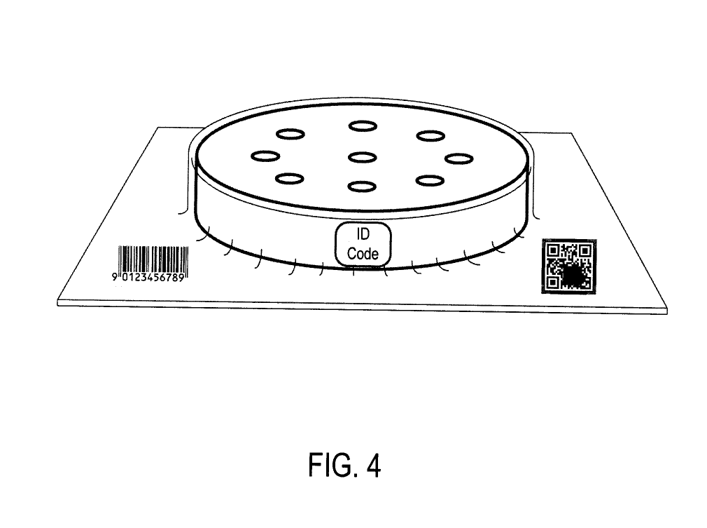

Now referring to FIG. 4, in another embodiment, the compressed vaporizer tablet may be individually sealed and packaged in blister packs. In another embodiment, the blister packs may be designed to be child resistant and/or senior friendly in order to increase safety and convenience. Furthermore, in addition to physically protecting the tablets, the blister packs and strictly control the amount of desired humidity in order to maintain freshness and increase longevity of the active ingredients.

As an illustrative example, each tablet may comprise approximately 1 gram of compressed plant source material. However, a larger or smaller amount of plant source material may be used. Furthermore, the size does not necessarily determine dosage, which may instead be controlled by an appropriate vaporizer machine used to vaporize the tablet.

In another embodiment, each tablet may include a label or a coating on at least one side of the tablet, which label or coating may include a barcode, or another type of machine readable code to identify the particular type of tablet. As a typical compressed vaporizer tablet in accordance with the present invention is significantly larger than pharmaceutical pills, tablets or capsules meant to be swallowed, the surface area of the compressed tablet (e.g. 15 mm-25 mm diameter) is sufficiently large for including a label or code.

Advantageously, the compressed vaporizer tablet of the present invention prevents degradation of the plant source material from friction and bruising. As well, the packaging of the blister packs increases safety and convenience, and increases the shelf life of the compressed plant source material by atmospherically sealing each tablet individually. Based on testing of shelf life under recommended storage conditions, an expiry date may be calculated and stamped or printed on each individual blister package.

Thus, in an aspect, there is provided a method of forming a compressed vaporizer tablet, comprising: obtaining one or more plant source materials containing one or more active medicinal ingredients; compressing the one or more plant source materials under pressure into a compressed vaporizer tablet; and forming one or more through holes in the compressed vaporizer tablet, whereby the through holes provide increased surface area in the compressed vaporizer tablet during vaporization.

In an embodiment, the method further comprises: heating lignin and trichomes present in the one or more plant source materials before compressing; and cooling the compressed vaporizer tablet after compressing to bind the one or more plant source materials.

In another embodiment, the method further comprises preparing the one or more plant source materials by one or more of drying, shredding and grinding.

In another embodiment, the method further comprises mixing the one or more plant source materials with one or more base materials for biding the plant source materials together during compression.

In another embodiment, the method further comprises selecting and blending the one or more plant source materials to achieve a desired blend of one or more active medicinal ingredients in the compressed vaporizer tablet.

In another embodiment, the method further comprises adding one or more flavoring agents, such that the compressed vaporizer tablet may deliver different aromatic flavors.

In another embodiment, the method further comprises forming the one or more through holes in the compressed vaporizer tablet with compression molds having at least one post or core.

In another embodiment, the method further comprises forming the one or more through holes in the compressed vaporizer tablet in a subsequent, separate drilling operation.

In another embodiment, the method further comprises forming ribbed edges along the outer side of the compressed vaporizer tablet.

In another embodiment, the method further comprises forming an alignment feature to align the one or more through holes in the compressed vaporizer tablet with a plurality of vents in a vaporizer.

In another embodiment, the method further comprises incorporating one or more machine identifiable features in the compressed vaporizer tablet, for identifying the type of compressed vaporizer tablet.

In another embodiment, the method further comprises individually sealing the compressed tablet in blister packaging, and incorporating one or more machine readable codes on the blister packaging for identifying the type of compressed vaporizer tablet.

In another embodiment, the one or one or more plant source materials includes at least one of hemp and cannabis.

In another aspect, there is provided a compressed vaporizer tablet, comprising: one or more plant source materials containing one or more active medicinal ingredients compressed under pressure; wherein, the compressed vaporizer tablet includes one or more through holes, thereby to increase the tablet surface area during vaporization.

In an embodiment, the compressed vaporizer tablet further comprises one or more base materials used for biding the plant source materials together.

In another embodiment, the one or more plant source materials are blended to achieve a desired blend of one or more active medicinal ingredients in the compressed vaporizer tablet.

In another embodiment, the compressed vaporizer tablet further comprises one or more flavoring agents, such that the compressed vaporizer tablet may deliver different aromatic flavors.

In another embodiment, the compressed vaporizer tablet further comprises ribbed edges formed along the outer side of the compressed vaporizer tablet.

In another embodiment, the compressed vaporizer tablet further comprises an alignment feature to align the one or more through holes in the compressed vaporizer tablet with a plurality of vents in a vaporizer.

In another embodiment, the compressed vaporizer tablet further comprises one or more machine identifiable features in the compressed vaporizer tablet, for identifying the type of compressed vaporizer tablet.

While illustrative embodiments of the invention have been described above, it will be appreciate that various changes and modifications may be made without departing from the scope of the present invention. For example, while the tablet has been shown as a relatively flat, wide cylinder, it will be appreciated that this shape is not limiting. Alternatively, the tablet may be an elongated cylindrical shape which may obviate the need for through holes by increasing the surface area relative to the mass of the tablet.

### REFERENCES

1. Institute of Medicine. Cannabinoids and animal physiology. Marijuana and medicine: Assessing the science base. Joy, J. E., Watson, S. J., and Benson, J. A. Washington, D.C.: National Academy Press, 1999.
2. De Petrocellis, L., Ligresti, A., Moriello, A. S., Allara, M. and others. (2011). Effects of cannabinoids and cannabinoid-enriched Cannabis extracts on TRP channels and endocannabinoid metabolic enzymes. Br. J. Pharmacol. 163: 1479-1494.
3. Izzo, A. A., Borrelli, F., Capasso, R., Di, Marzo, V and others. (2009). Non-psychotropic plant cannabinoids: new therapeutic opportunities from an ancient herb. Trends Pharmacol. Sci. 30: 515-527.
4. Musty, R. E. Natural cannabinoids: interactions and effects. The medicinal uses of cannabis and cannabinoids. Guy, G. W, Whittle, B. A., and Robson, P. J. London: Pharmaceutical Press, 2004.
5. Health Canada. Information for Health Care Professionals: Daily Amounts Fact Sheet. Available at: http://www.hc-sc.gc.ca/dhp-mps/marihuana/med/index-eng.php
6. M A Ware, H Adams, and GW Guy. “The medicinal use of cannabis in the UK: results of a nationwide survey,” Int. J. Clin. Pract. 2005. 59:291-295.
7. Pertwee, R. G. (2008). The diverse CB1 and CB2 receptor pharmacology of three plant cannabinoids: delta9-tetrahydrocannabinol, cannabidiol and delta9-tetrahydrocannabivarin. Br. J. Pharmacol. 153: 199-215.
8. G T Carter, P Weydt, M Kyashna-Tocha and DI Abrams. “Medicinal cannabis: rational guidelines for dosing,” IDrugs. 2004. 7:464-470.
9. A J Clark, M A Ware, E Yazer, T J Murray et al. “Patterns of cannabis use among patients with multiple sclerosis,” Neurology. 2004. 62:2098-2100.
10. M A Ware, T Wang, S Shapiro, A Robinson et al. (2010) Smoked Cannabis for Chronic Pain: a Randomized Controlled Trial. Canadian Medical Association Journal (CMAJ). 182:E694-E701

1. A method of forming a compressed vaporizer tablet, comprising:
obtaining one or more plant source materials containing one or more active medicinal ingredients;
compressing the one or more plant source materials under pressure into a compressed vaporizer tablet; and
forming one or more through holes in the compressed vaporizer tablet, whereby the through holes provide increased surface area in the compressed vaporizer tablet during vaporization.

2. The method of claim 1, further comprising:
heating lignin and trichomes present in the one or more plant source materials before compressing; and
cooling the compressed vaporizer tablet after compressing to bind the one or more plant source materials.

3. The method of claim 1, further comprising preparing the one or more plant source materials by one or more of drying, shredding and grinding.

4. The method of claim 3, further comprising mixing the one or more plant source materials with one or more base materials for biding the plant source materials together during compression.

5. The method of claim 3, further comprising selecting and blending the one or more plant source materials to achieve a desired blend of one or more active medicinal ingredients in the compressed vaporizer tablet.

6. The method of claim 3, further comprising adding one or more flavoring agents, such that the compressed vaporizer tablet may deliver different aromatic flavors.

7. The method of claim 1, further comprising forming the one or more through holes in the compressed vaporizer tablet with compression molds having at least one post or core.

8. The method of claim 1, further comprising forming the one or more through holes in the compressed vaporizer tablet in a subsequent, separate drilling operation.

9. The method of claim 1, further comprising forming ribbed edges along the outer side of the compressed vaporizer tablet.

10. The method of claim 1, further comprising forming an alignment feature to align the one or more through holes in the compressed vaporizer tablet with a plurality of vents in a vaporizer.

11. The method of claim 1, further comprising incorporating one or more machine identifiable features in the compressed vaporizer tablet, for identifying the type of compressed vaporizer tablet.

12. The method of claim 1, further comprising individually sealing the compressed tablet in blister packaging, and incorporating one or more machine readable codes on the blister packaging for identifying the type of compressed vaporizer tablet.

13. The method of claim 1, wherein the one or one or more plant source materials includes at least one of hemp and cannabis.

14. A compressed vaporizer tablet, comprising:
one or more plant source materials containing one or more active medicinal ingredients compressed under pressure;
wherein, the compressed vaporizer tablet includes one or more through holes, thereby to increase the tablet surface area during vaporization.

15. The compressed vaporizer tablet of claim 14, further comprising one or more base materials used for biding the plant source materials together.

16. The compressed vaporizer tablet of claim 14, wherein the one or more plant source materials are blended to achieve a desired blend of one or more active medicinal ingredients in the compressed vaporizer tablet.

17. The compressed vaporizer tablet of claim 14, further comprising one or more flavoring agents, such that the compressed vaporizer tablet may deliver different aromatic flavors.

18. The compressed vaporizer tablet of claim 14, further comprising ribbed edges formed along the outer side of the compressed vaporizer tablet.

19. The compressed vaporizer tablet of claim 14, further comprising an alignment feature to align the one or more through holes in the compressed vaporizer tablet with a plurality of vents in a vaporizer.

20. The compressed vaporizer tablet of claim 14, further comprising one or more machine identifiable features in the compressed vaporizer tablet, for identifying the type of compressed vaporizer tablet.
